(19) United States
(12) Patent Application Publication
(10) Pub. No.: US 2017/0325718 A1
(43) Pub. Date: Nov. 16, 2017

(54) Neuropathic Diagnosis and Monitoring Using Earpiece Device, System, and Method

(71) Applicant: BRAGI GmbH, München (DE)

(72) Inventors: Peter Vincent Boesen, München (DE); Friedrich Christian Förstner, München (DE); Martin Steiner, München (DE)

(73) Assignee: BRAGI GmbH, München (DE)

(21) Appl. No.: 15/592,839

(22) Filed: May 11, 2017

Related U.S. Application Data
(60) Provisional application No. 62/335,217, filed on May 12, 2016.

(51) Int. Cl. A61B 5/11; A61B 5/00

(52) U.S. Cl. CPC A61B 5/1101; A61B 5/4082; A61B 5/0082; A61B 5/6803; A61B 5/0015; A61B 5/1126; A61B 2560/0242; A61B 2562/0219

(57) ABSTRACT

A method of diagnosing and monitoring biologic dysfunction may include performing measurements utilizing sensors of wireless earpieces, analyzing the measurements, comparing the measurements to established norms, determining whether the measurements indicate biologic dysfunction. The measurements may include biologic data of a user or environmental data. The sensors may include inertial sensors or optical sensors. The biologic dysfunction may include neurologic dysfunction and the neurologic dysfunction may include intention tremor. The method may include reporting the biologic dysfunction to a user.

### PRIORITY STATEMENT

This application claims priority to U.S. Provisional Patent Application 62/335,217, filed on May 12, 2016, and entitled Neuropathic Diagnosis and Monitoring Using Earpiece Device, System, and Method, hereby incorporated by reference in its entirety.

### BACKGROUND

### I. Field of the Disclosure

The illustrative embodiments relate to wearable devices. More particularly, but not exclusively, the illustrative embodiments relate to earpieces.

### II. Description of the Art

The growth of wearable devices is increasing exponentially. This growth is fostered by the decreasing size of microprocessors, circuity boards, chips, and other components. The ear and ear canal provide a potentially rich environment for the collection of biometric data through the use of wearable devices and, particularly, earpieces. This is, in part, because the external ear canal sits in close proximity to the central nervous system. In addition to being positioned near the central nervous system, the positioning of the ear canal on the head and near the neck makes it a desirable place for the detection of movement in the head and neck area. In certain instances, detectable movements in the head and neck, for example, intention tremors, are indicative of neurologic diseases or defects. The use of sensors contained in wearable devices, particularly earpieces, can assist in the diagnosis and monitoring of such neurologic disease. Comparison of biologic data retrieved from the sensors can also be utilized to interact with and modify the user's personal environment.

### SUMMARY OF THE DISCLOSURE

Therefore, it is a primary object, feature, or advantage to improve over the state of the art.

It is a further object, feature, or advantage to diagnose neurologic diseases or defects through the monitoring of biologic data.

It is a further object, feature, or advantage to detect intention tremor through the monitoring of biologic data.

It is a still further object, feature, or advantage to analyze biologic data detected through sensors for the purpose of communicating said data to devices in the user's personal area network for comparison and analysis.

Yet another object, feature, or advantage is to determine different states of the user by combining and comparing biologic data obtained through the sensors with data obtained through the user's personal area network.

Yet another object, feature, or advantage is to utilize biologic data obtained from the sensor's and the user's personal area network to provide the user with feedback related to such data.

One or more of these and/or other objects, features, or advantages will become apparent from the specification and claims that follow. No single embodiment need provide each or every one of these objects, features, or advantages. Instead, different embodiments may have different objects, features, or advantages. The present invention is not to be limited by or to these objects, features, and advantages.

According to one aspect a method for diagnosing and monitoring neurologic dysfunction is provided. The method may include performing measurements utilizing sensors of wireless earpieces, analyzing the measurements, comparing the measurements to established norms, determining whether the measurements indicate neurologic dysfunction. The measurements may include biologic data of a user or environmental data. The sensors may include an accelerometer and a gyrometer. The neurologic dysfunction may include intention tremor. The method may include reporting the neurologic dysfunction to a user.

According to another aspect a method for diagnosing and monitoring neurologic dysfunction is provided. The method may include performing a first set of measurements utilizing a first set of sensors of wireless earpieces, performing a second set of measurements utilizing a second set of sensors of electronic devices, analyzing the first and second set of measurements, comparing the first and second set of measurements to established norms, and determining whether the measurements indicate neurologic dysfunction. The first and second set of measurements may include biologic data of a user or environmental data. The sensors of the wireless earpiece may include an accelerometer and a gyrometer. The neurologic dysfunction may include intention tremor. The method may include reporting the neurologic dysfunction to a user.

### BRIEF DESCRIPTION OF THE DRAWINGS

Illustrated embodiments of the present invention are described in detail below with reference to the attached drawing figures, which are incorporated by reference herein, and where:

FIG. 1 is a pictorial representation of a communication system in accordance with an illustrative embodiment;

FIG. 2 is a pictorial representation of a diagnosis and monitoring system in accordance with an illustrative embodiment;

FIG. 3 is a block diagram of a diagnosis and monitoring system in accordance with an illustrative embodiment;

FIG. 4 is a diagram illustrating one example of a method.

FIG. 5 is another diagram of a method.

### DETAILED DESCRIPTION OF THE DISCLOSURE

The illustrative embodiments provide a device, system, and method for neuropathic diagnosis and monitoring. The electronics package of wearable devices may contain sensors including temperature sensors, pulse oximeters, accelerometers, gyroscopes, altitude sensors, GPS chips, and so forth. The sensors may be utilized to sense any number of biometric readings or information, such as heart rate, respiratory rate, blood, or skin physiology, body movement, or other biometric data. Advantageous locations for the placement of such sensors and wearable devices may be locations that can easily access biometric data without interfering with the lifestyle of the user. These sensors and wearable devices and the information that they collect can be used to report important personal health information back to the user and those that user chooses to provide access.

In one embodiment, the electronic device is one or more wireless earpieces. Generally, the wireless earpieces may be utilized to make and receive communications (e.g., telephone calls, transcribed text messages, audio/tactile alerts, etc.), play music, filter or block sound, amplify sounds, or so forth. In addition to these functions, the wireless earpiece may be fitted with biologic sensors to collect biologic data and information about the user. The external auditory canal sits in close proximity to the central nervous system, making it a good location for the placement of sensors and collection of biologic data. Additionally, the positioning of the ear on the head makes it a good location for the collection of data related to head and neck movements.

Wearable devices placed in the ear and the auditory canal can be fitted with sensors including, for example, accelerometers and gyroscopes, that can detect and monitor movements in the head and neck. The sensors may be stand-alone measurement devices or may be integrated in one or more chips, motherboards, cards, circuits, or so forth. The integration of these measurement devices, and particularly those that measure biologic data, can be used, for example, to monitor and detect abnormalities or departures from normal biologic functioning.

One such abnormality is neurological diseases, or diseases of the brain, spine, and central nervous system. The onset of neurological diseases is oftentimes marked by a slow and steady progression of symptoms until the symptoms become clinically obvious. One sign or symptom of neurological diseases is the onset and persistence of intention tremors. Intention tremor, often referred to as cerebellar tremor, is a dyskinetic disorder characterized by broad, coarse, and low frequency undulations at less than 5 Hz. The most common cause of persistent intention tremor involves damage or degeneration from the cerebellum. This damage can take multiple forms for example, a cerebellar lesion. One function of the cerebellum is to receive information from the sensory systems, spinal cord, and brain and utilize that information to regulate motor movements. The typical site of cerebellar lesions leading to persistent intention tremor is the cerebellar peduncle, where the fibers traversing the peduncle transmit information to the midbrain, as well as the dentate nucleus.

The causes of intention tremor are many including, for example, alcohol abuse, multiple sclerosis, Wilson's disease of the liver, and hypoparathyroidism. Poisons—such as mercury—vitamin deficiencies—such as Vitamin E—and pharmalogic medications—such as cyclosporine, lithium, and stimulants—can cause intention tremor. Alcohol abuse, for example, can lead to damage of the cerebellum over time, leading to the degradation of control over fine motor movement. Such degradation over time manifests in patient inability to adequately process fine motor control, leading to the clinical phenomenon of intention tremors.

The use of an earpiece with sensors, for example accelerometers and gyrometers capable of detecting fine motor control dysfunction can be positioned at the external auditory canal of a user or patient, which is a desirable location for the detection and monitoring of intention tremor. Using these sensors can allow distinction and segregation of intention tremor from resting tremors based on further accelerometer data inputs related to the user and user's environment. This system provides a non-invasive system which is able to detect fine motor control dysfunction both from a primary detection phase, as well as providing ongoing input as to its status and progression.

Such a system would be able to utilize the sensor data, for example the data from the accelerometer, in order to perform a comparative analysis to established norms. The data could be derived from the device in an ongoing fashion in order to gauge the progression of such an issue. Such data could also be used in situations to gauge the onset of intention tremor and its diminution over time in alternative states, such as, for example, stress, increased anxiety, fatigue, fear, or anger. These differing states can be detected and determined by utilizing the multiple associated sensors at the earpiece, for example, sweat sensors, microphones, and heart rate sensors. These types of intention tremors could themselves be utilized to gauge the emotional status of the user. In turn, feedback responses or other results could be produced based on the cumulative analysis of the biometric data.

For example, if an intention tremor is detected and associated biometric sensors indicate a raised voice (microphone inputs) elevated heart rate (heart rate sensors), and increased sweating (sweat sensors), such factors would be analyzed and would be indicative of a high stress level. Another example would be the detection of jitter (a measure of period-to-period fluctuations in fundamental frequency) and shimmer (a measure of period-to-period variability in amplitude values) levels could be employed through the use of the microphone inputs to detect and compare abnormalities in the user's biologic data. The device would be able to interact with the user to alert them of the changes in said parameters, and possibly to suggest alternative behaviors. If the intention tremors persist over time, algorithms employed by the device would be able to determine the length and progression of such phenomena in order to alert the user of its presence. The sensor arrays could also be utilized to monitor changes in the user's posture, adding to a comprehensive picture of the user's overall status.

The earpiece sensors and biologic data can be combined with additional data or environmental information to assess and inform the user of a myriad of differing assessments and indications. For example, if intention tremors are detected by the earpiece, other sensor groups (for example, one detecting alcohol levels in the bloodstream) could be used in combination with the intention tremor measurements to determine the level of impairment of the user. Feedback to the user could then be facilitated, as well as other devices within the user's personal area network. This would be especially useful in situations, for example, where said earpiece user was also driving a connected automobile or vehicle. Alerts, or even electronic disablement of the vehicle could result from such data, potentially saving the user or others from serious harm or dysfunction. There are multiple other use cases from positioning sensitive accelerometric sensors at or in the user's external auditory canal. These devices would be of great utility in the diagnosis, monitoring, and management of numerous physiologic and pathologic states. In addition to collecting data, the wireless earpieces may communicate with other wearables (e.g., smart watch, ring, jewelry, smart wearables, etc.) to modify, filter, or otherwise optimize the accuracy of sensor measurements.

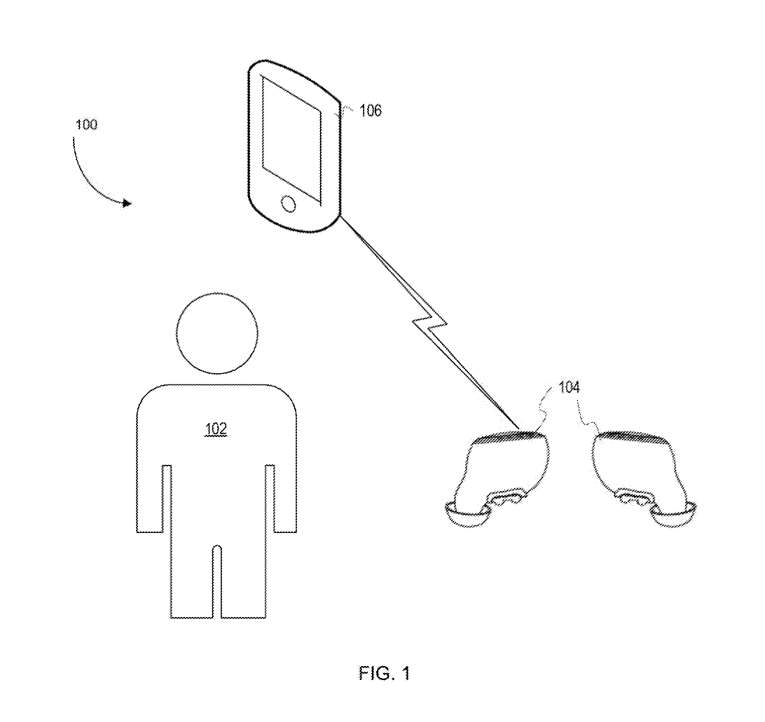

FIG. 1 is a pictorial representation of a communications system 100 in accordance with an illustrative embodiment. In one embodiment, the communication system 100 may be used by a user 102 and may include wireless earpieces 104, and a wireless device 106. The wireless earpieces 104 may be referred to as a pair or set (wireless earpieces 104) or singularly (wireless earpiece). In one embodiment, the wireless earpieces 104 include a left earpiece and a right earpiece configured to fit into a user's 102 ears. The wireless earpieces 104 are shown separately from their positioning within the ears of the user 102 for purposes of simplicity. The wireless earpieces 104 may be configured to play music or audio, receive and make phone calls or other communications, determine ambient environmental readings (e.g., temperature, altitude, location, speed, heading, etc.), read user biometrics and actions (e.g., heart rate, motion, sleep, blood oxygenation, calories burned, etc.).

The wireless earpieces 104 may include interchangeable parts that may be adapted to fit the needs of the user 102. For example, sleeves that fit into the ear of the user 102 may be interchangeable to find a suitable shape and configuration. The wireless earpieces 104 may include a number of sensors and input devices including, but not limited to, pulse oximeters, microphones, pulse rate monitors, accelerometers, gyroscopes, light sensors, global positioning sensors, and so forth. Sensors of the wireless device 106 may also be configured to wirelessly communicate with the wireless earpieces 104.

The wireless device 106 may represent any number of wireless electronic devices, such as smart phones, laptops, gaming devices, music players, personal digital assistants, vehicle systems, or so forth. The wireless device 106 may communicate utilizing any number of wireless connections, standards, or protocols (e.g., near field communications, Bluetooth, Wi-Fi, ANT+, etc.). For example, the wireless earpieces 104 may be a touch screen cellular phone that communicates with the wireless device 106 utilizing Bluetooth communications. The wireless device 106 may implement and utilize any number of operating systems, kernels, instructions, or applications that may make use of the sensor data measured by the wireless earpieces 104. For example, the wireless device 106 may represent any number of android, iOS, Windows, open platform, or other systems. Similarly, the wireless device 106 may include a number of applications that utilize the biometric data from the wireless earpieces 104 to display applicable information and data. For example, the information (including, high, low, average, or other values) may be processed by the wireless earpieces 104 or the wireless device 106 to display heart rate, blood oxygenation, altitude, speed, distance traveled, calories burned, or other applicable information.

Sensor measurements made by either the wireless earpieces 104, wireless device 106, or sensor devices of the user 102 may be communicated with one another in the communication system 100. The measurements made by the wireless device 106, or other sensor devices of the user 102, may be used to inform the user of changes in biologic measurements. The wireless device 106 is representative of any number of personal computing, communications, exercise, medical, or entertainment devices that may communicate with the wireless earpieces 104.

The user 102 may also be wearing or carrying any number of sensor-enabled devices, such as heart rate monitors, pacemakers, smart glasses, smart watches or bracelets (e.g., Apple watch, Fitbit, etc.), or other sensory devices that may be worn, attached to, or integrated with the user 102. The data and information from the external sensor devices may be communicated to the wireless earpieces 104.

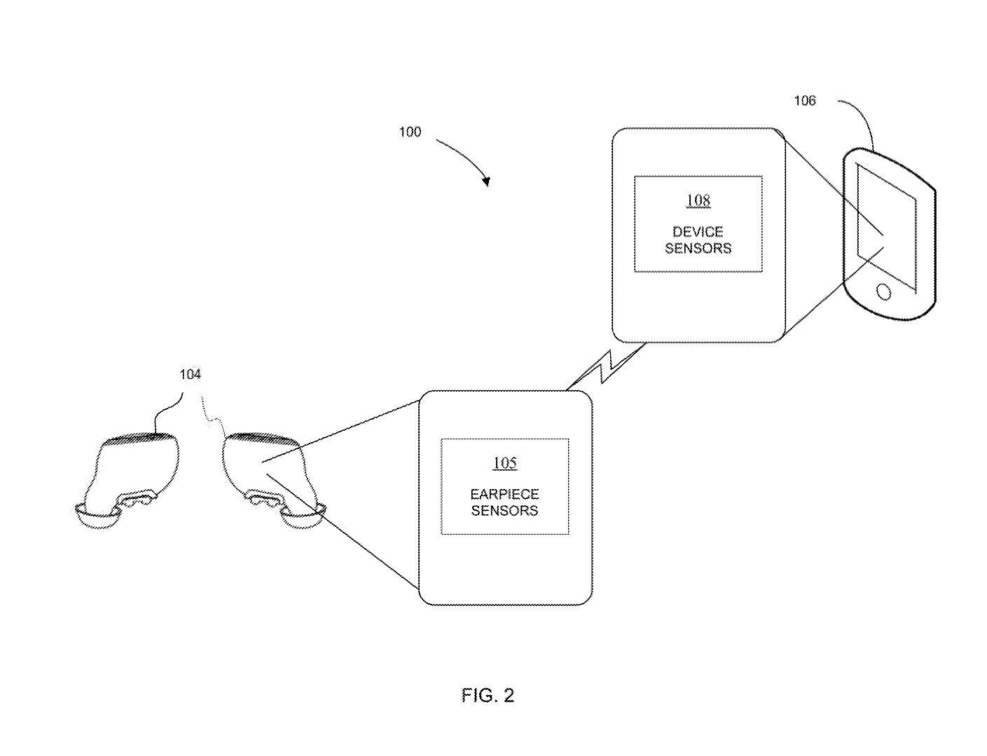

FIG. 2 is a pictorial representation of a diagnosis and monitoring system 100 in accordance with an illustrative embodiment. The wireless earpieces 104 are equipped with at least one of a variety of earpiece sensors 105, including, but not limited to, pulse oximeters, microphones, pulse rate monitors, accelerometers, gyroscopes, light sensors, global positioning sensors, and so forth. The earpiece sensors of the wireless earpieces 104 may be configured to wirelessly communicate with a wireless device 106 in the user's personal area network. The wireless device 106 is equipped with at least one of a variety of device sensors 108. Device sensors 108 of the wireless device 106 may also be configured to wirelessly communicate with the wireless earpieces 104. The data and measurements from the device sensors 108 may be combined with the data and measurements from the earpiece sensors 105 to determine biologic function of the user. For example, the data and measurements from the device sensors 108 may be combined with the data from the earpiece sensors 105 and compared to established norms for the detection of neurologic dysfunction including, for example, intention tremors. It is to be understood, however, that any number of different profiles may be established associated with different dysfunctions. Thus, sensor measurements from the earpiece may be used to assist in diagnosis of a condition of a user by collecting sensor data. The sensor data may then be compared to the profiles to assist in diagnosis of the dysfunction. In addition to determining a type of a particular dysfunction, the sensor data may be used to assist in determining the severity of the dysfunction.

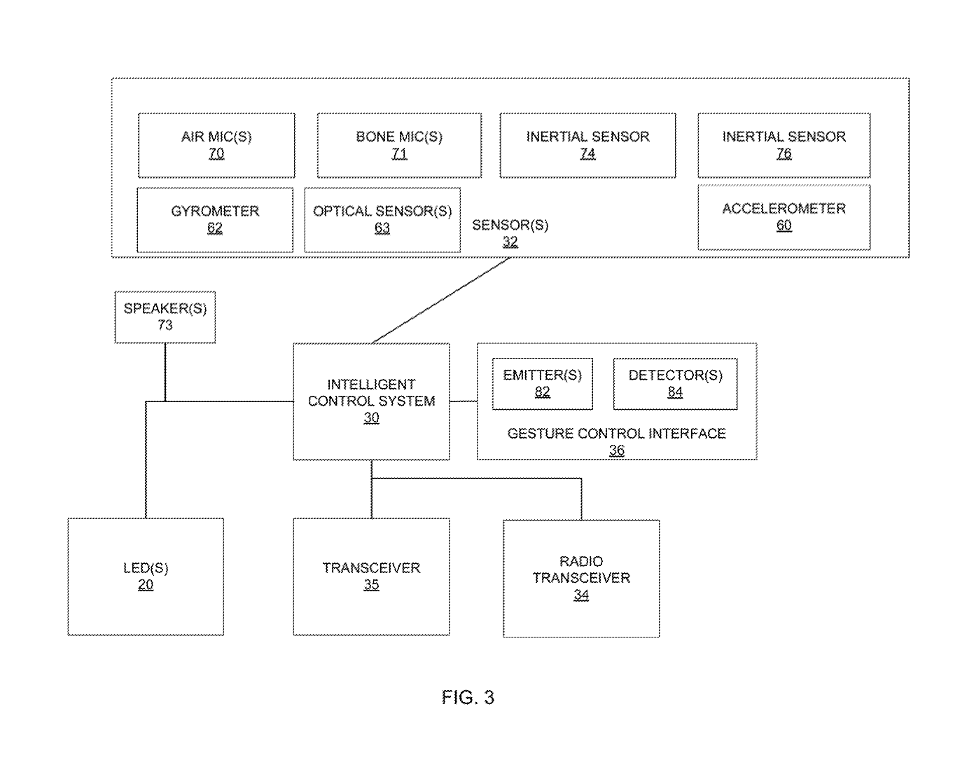

FIG. 3 is a block diagram illustrating an earpiece. The earpiece may include one or more sensors 32. The sensors may include one or more air microphones 70, one or more bone microphones 71, and one or more inertial sensors 74, 76, one or more optical sensors 62, or other types of sensors. Each of the one or more sensors 32 is operatively connected to an intelligent control system 30. The intelligent control system 30 may also be operatively connected to a gesture control interface 36 which may include one or more emitters 82 and one or more detectors 84. The gesture control interface 36 allows a user to interact with the earpiece through gestures or motions which are detected by the gesture control interface and interpreted by the intelligent control system 30. The emitters 82 and detectors 84 of the gesture control interface 36 may be optical emitters and detectors which in addition to detecting gestures of a user may also be used to detect tics, tremors, or other movements of the user indicative of a medical condition. One or more speakers 72 is operatively connected to the intelligent control system 30. One or more light emitting diodes 20 are operatively connected to the intelligent control system 30 that may be used to provide visual feedback indicative of earpiece functionality or status. A radio transceiver 34 is shown as well as a second transceiver 35 which may be an NFMI transceiver or other type of transceiver. An accelerometer 60 and gyrometer 62 may be operatively connected to the intelligent control system 30. Other types of inertial sensors 74, 76 may be used.

Where multiple earpieces (i.e. a left earpiece and a right earpiece) are used within a system it is contemplated that the left earpiece and the right earpiece may have different functionality. For example, not every sensor need be present in both earpieces, the different earpieces may have different amounts of processing capabilities, storage, or other features.

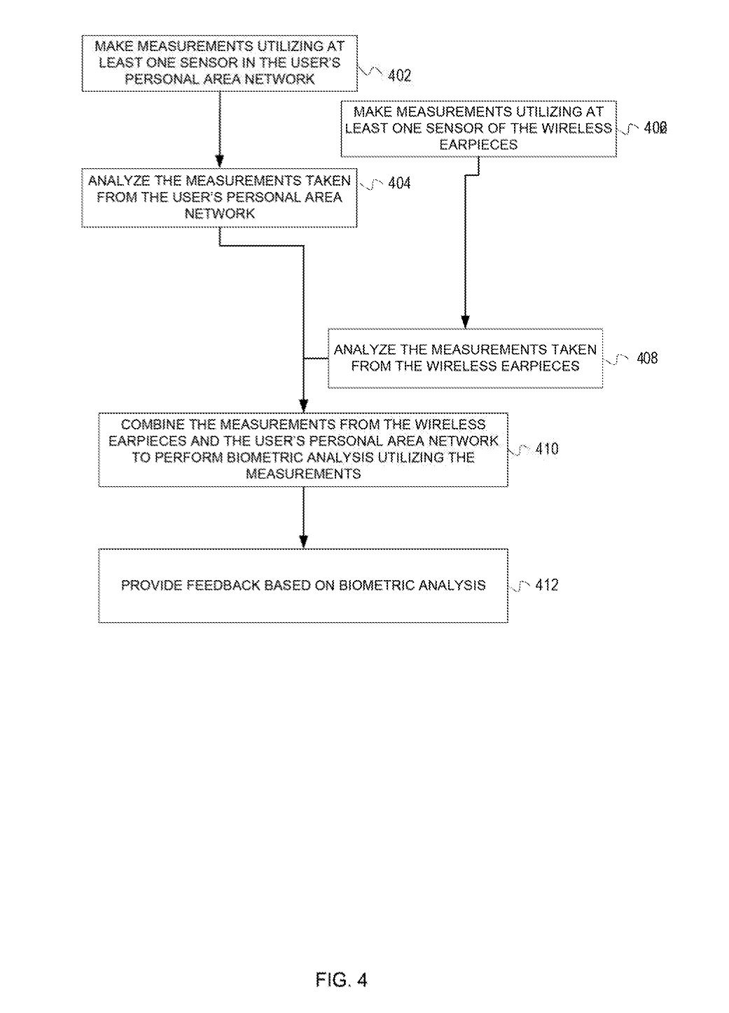

FIG. 4 illustrates one example of a method. In one embodiment, the diagnosis and monitoring system may make measurements utilizing at least one sensor in the user's personal area network (step 402). This may include utilizing electronic devices in the user's network or that are associated with the user including mobile devices, wearable devices and accessories. Once the measurements are collected, the measurements are analyzed (step 404). Additionally, the diagnosis and monitoring system may make measurements utilizing at least one sensor of the wireless earpieces (step 406) and analyze the measurements taken from the wireless earpieces (step 408). Once these measurements are analyzed, the diagnosis and monitoring system may combine the measurements from the wireless earpieces and the user's personal area network to perform biometric analysis utilizing the measurements (step 410). This combination of measurements can be used to provide feedback based on the biometric analysis (step 412).

For example, where inertial data is used, the magnitude of movements, the frequency, or intensity may be used to aid in the analysis. One or more of these characteristics may be used in the analysis. Where optical sensors are used, such as optical emitters and receives associated with the gesture control, facial tics, spasms, or other movements may be detected which are indicative of a disorder.

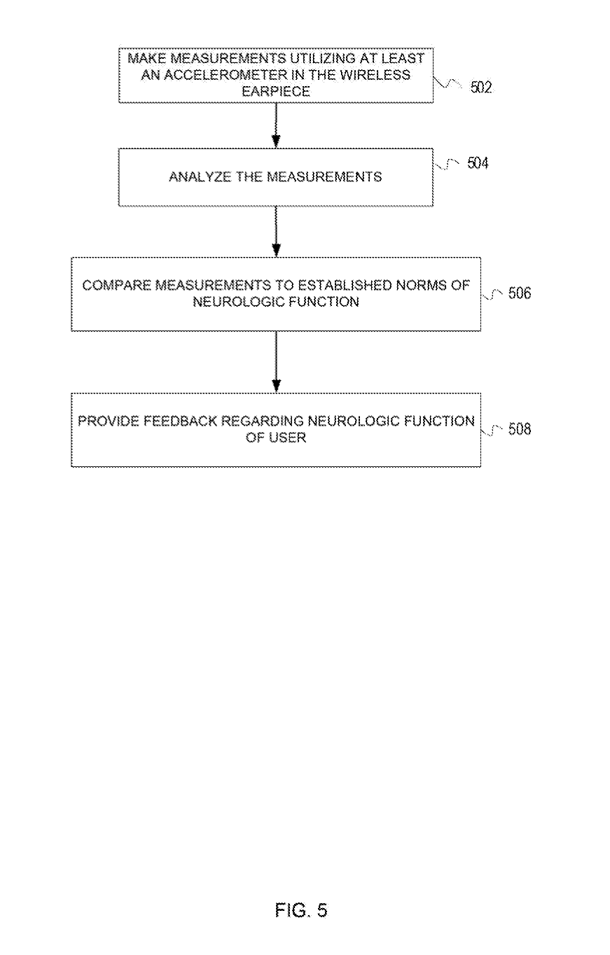

FIG. 5 illustrates another method. In one embodiment the diagnosis and monitoring system may make measurements utilizing at least an accelerometer in the wireless earpiece (step 502) and then analyze those measurements (step 504). Those measurements might be compared or combined with other measurements taken from additional sensors such as a gyrometer. Once the measurements are analyzed, the diagnosis and monitoring system 500 may compare the measurements to established norms of neurologic function (step 506) to diagnose, detect, and provide feedback regarding the neurologic function of the user (step 508).

The illustrative embodiments are not to be limited to the particular embodiments described herein. In particular, the illustrative embodiments contemplate numerous variations in the type of ways in which embodiments may be applied. The foregoing description has been presented for purposes of illustration and description. It is not intended to be an exhaustive list or limit any of the disclosure to the precise forms disclosed. It is contemplated that other alternatives or exemplary aspects are considered included in the disclosure. The description is merely examples of embodiments, processes or methods of the invention. It is understood that any other modifications, substitutions, and/or additions may be made, which are within the intended spirit and scope of the disclosure. For the foregoing, it can be seen that the disclosure accomplishes at least all of the intended objectives.

The previous detailed description is of a small number of embodiments for implementing the invention and is not intended to be limiting in scope. The following claims set forth a number of the embodiments of the invention disclosed with greater particularity.

What is claimed is:

1. A method for diagnosis and monitoring, the method comprising:
providing at least one earpiece comprising an earpiece housing, a processor disposed within the earpiece, and at least one sensor disposed within the housing and operatively connected to the processor;
performing measurements utilizing one or more of the sensors of the at least one wireless earpiece;
analyzing the measurements;
comparing the measurements to established norms; and
determining whether the measurements indicate neurologic dysfunction.

2. The method of claim 1, wherein the steps of analyzing the measurements are performed by the processor disposed within the earpiece housing.

3. The method of claim 1 wherein the at least one earpiece comprises a wireless transceiver disposed within the housing and operatively connected to the processor.

4. The method of claim 3 further comprising sending the measurements from the earpiece to a remote device using the transceiver.

5. The method of claim 4 wherein the at least one sensor includes at least one inertial sensor.

6. The method of claim 5, wherein the at least one inertial sensor includes at least an accelerometer and a gyrometer.

7. The method of claim 6, wherein the measurements include fine motor movements.

8. The method of claim 7, wherein the neurologic dysfunction is an intention tremor dysfunction.

9. The method of claim 1, further comprising:
notifying a user of the neurologic dysfunction.

10. The method of claim 1, wherein the measurements include at least one environmental measurement.

11. A method for diagnosis and monitoring, the method comprising:
performing a first set of measurements utilizing a first set of sensors of wireless earpieces;
performing a second set of measurements utilizing a second set of sensors of electronic devices;
analyzing the first and second set of measurements;
comparing the first and second set of measurements to established norms; and
determining whether the measurements indicate neurologic dysfunction.

12. The method of claim 11, wherein the first set of measurements are biometric measurements of a user utilizing the wireless earpieces.

13. The method of claim 11, wherein the first set of measurements are environmental measurements.

14. The method of claim 13, wherein the second set of measurements are biometric measurements of a user utilizing the electronic device.

15. The method of claim 11, wherein the first set of sensors include at least one inertial sensor.

16. The method of claim 15 wherein the at least one inertial sensor includes an accelerometer and a gyrometer and wherein the first set of measurements are fine motor movements.

17. The method of claim 16, wherein the neurologic dysfunction is an intention tremor dysfunction.

18. The method of claim 11, further comprising notifying a user of the neurologic dysfunction.

19. The method of claim 11 wherein the first set of sensors are optical sensors.

20. The method of claim 19 wherein the optical sensors are used in a gesture control interface of the wireless earpieces.
